(19) United States
(12) Patent Application Publication
(10) Pub. No.: US 2007/0185431 A1
(43) Pub. Date: Aug. 9, 2007

(54) Galvanic Current Skin Treatment

(76) Inventor: Dale G. Kern, Hyde Park, UT (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/670,643

(22) Filed: Feb. 2, 2007

Related U.S. Application Data
(60) Provisional application No. 60/765,397, filed on Feb. 3, 2006.

(51) Int. Cl. A61N 1/30

(52) U.S. Cl. 604/20

(57) ABSTRACT

The present invention relates to methods and systems for application of a substance and an electrical current to a biological tissue or surface. In one embodiment, the methods and systems include application of a substance and an electrical current to the surface or tissue such that the transport or absorption of the substance into the surface or tissue is enhanced for a period of time after the electrical current has been removed.

### CROSS-REFERENCE TO RELATED APPLICATION(S)

The instant application claims priority to U.S. Provisional Application 60/765,397, which is hereby incorporated herein by reference in its entirety.

### FIELD OF THE INVENTION

The present invention relates to the application of a substance and an electrical current to a biological tissue or surface, and more specifically to a system and method for enhancing the transportation, absorption, or other type of intake of the substance into the biological surface or tissue upon and after the application of the electrical current.

### BACKGROUND OF THE INVENTION

The process of iontophoresis was described in 1908, and has since found commercial use in the delivery of ionically charged compounds. In this delivery method, ions bearing positive and negative charges may be driven across the skin at the sites of electrical nodes.

Iontophoretic instruments essentially include an electrode attached to a patient, each connected by a wire to a microprocessor to control the electrical instrument. Examples of such instruments are described in U.S. Pat. Nos. 5,254,081, and 5,431,625. Power for these instruments is usually provided by DC batteries, which when providing power to the microprocessor circuitry allows application of a voltage to the electrodes to create a regulated current flow.

Iontophoretic drug delivery systems of the prior art have been limited primarily to delivering a drug of a single polarity at a time to a given area in relatively low concentrations and have not been suitable for simultaneous administration of multiple drugs. In addition, the duration of effective treatment derived from a particular electroconnetic treatment has heretofore been limited to the actual duration over which current is applied to a person.

There is a need in the art, therefore, for methods, systems, and devices that improve over prior iontophoretic procedures.

### BRIEF SUMMARY

One embodiment disclosed herein is a method of treating skin with an electrical current. The method includes applying a substance to the skin and applying an electrical current to the skin for a predetermined amount of time. In this method, the transport or absorption of the substance into the skin is enhanced for a period of time after terminating the electrical current. In one embodiment, the method can also include allowing the substance to remain in contact with the skin for a predetermined period of time after terminating the electrical current.

Another embodiment is a method of treating a biological surface with an electrical current. The method includes applying a substance to the surface, applying an electrical current to the surface for a predetermined amount of time, and allowing the substance to remain in contact with the surface for a predetermined period of time after terminating the electrical current. In this method, transport or absorption of the substance into the surface is enhanced for a period of time after terminating the electrical current.

A further implementation relates to a device for testing application of electrical current to a cellular membrane. The device includes a Franz cell, positive and negative electrodes operably coupled to the Franz cell, and an electrical current application device operably coupled to the positive and negative electrodes.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a schematic drawing of a prior art Franz cell.

FIG. 2 is a schematic drawing of a cellular membrane test device, according to one embodiment.

FIG. 3A is a line graph depicting the amount of caffeine that penetrated human skin over time after application of an electrical current to the skin, according to one embodiment.

FIG. 3B is a bar graph depicting the amount of caffeine that penetrated human skin 24 hours after application of an electrical current to the skin, according to one embodiment.

FIG. 4A is a bar graph depicting the amount of caffeine that penetrated human skin at various levels of electrical current 6 hours after application of an electrical current to the skin, according to one embodiment.

FIG. 4B is a line graph depicting the amount of caffeine that penetrated human skin at various levels of electrical current 6 hours after application of an electrical current to the skin, according to one embodiment.

FIG. 5A is a bar graph depicting the amount of caffeine that penetrated human skin at two levels of electrical current 6 hours after one application of electrical current to the skin, according to one embodiment.

FIG. 5B is a bar graph depicting the amount of caffeine that penetrated human skin at two levels of electrical current at 24 hours, wherein the lower level of electrical current was applied twice, according to one embodiment.

FIG. 5C is a line graph depicting the amount of caffeine that penetrated human skin at two different levels of electrical current over time, wherein the lower level of electrical current was applied twice, according to one embodiment.

FIG. 6 is a line graph depicting the amount of caffeine that penetrated human skin when a single application of electrical current was compared to a double application of electrical current over time, according to one embodiment.

FIGS. 7A, 7B, 7C, and 7D are bar graphs depicting the amount of caffeine that penetrated human skin when a single application of electrical current was compared to a double application of electrical current, according to one embodiment.

FIG. 7E is a line graph depicting the amount of caffeine that penetrated human skin when a single application of electrical current was compared to a double application of electrical current over time, according to one embodiment.

FIGS. 8A and 8B are line graphs depicting the correlation between the amount of caffeine that penetrated human skin and the amount of electrical current, according to one embodiment.

FIGS. 9A, 9B, and 9C are bar graphs depicting the amount of aminophylline that penetrated human skin at different levels of electrical current over time, according to one embodiment.

FIG. 10 is a bar graph depicting the amount of theobromine that penetrated human skin at different levels of electrical current 24 hours after application of the electrical current, according to one embodiment.

FIGS. 11A and 11B are a bar graph and line graph, respectively, depicting the amount of ethyl nicotinate that penetrated human skin over time after application of an electrical current, according to one embodiment.

FIG. 12 is a bar graph depicting the amount of thiotaine that penetrated human skin over time after application of an electrical current, according to one embodiment.

FIG. 13A is a chart describing the percentage of subjects that exhibited a reduction in thigh diameter after 8 weeks using a body shaping gel with application of electrical current, according to one embodiment.

FIG. 13B is a line graph depicting amount of cellulite reduction in subjects over time, according to one embodiment.

FIG. 13C is a pie graph depicting percentage of subjects who exhibited one grade level or more improvement in the amount of visible cellulite, according to one embodiment.

FIG. 13D is a chart describing percentage of subjects showing improvement after 8 weeks of using body shaping gel with application of electrical current, according to one embodiment.

FIGS. 14A, 14B, and 14C are charts showing results of examination of LED levels at which subjects first noticed any sensation or felt discomfort on various body parts, according to one embodiment.

FIG. 14D is a chart showing electrical current levels of a galvanic current application device, according to one embodiment.

### DETAILED DESCRIPTION

The present invention relates to systems and methods for application of electrical energy to a suitable surface of a biological subject, such as the skin of a human body. According to certain embodiments, the method or system includes applying electrical energy to the surface in combination with administration of a medicament and can further include maximizing the effects of the medicament, including increasing the intake of the medicament into or through the surface for some period of time after the application of electrical energy has ceased. Alternatively, the application of electrical energy relates to other electrotherapeutic treatments.

In accordance with one implementation, the method, system, or apparatus relates to transdermal delivery or transport of a substance using an iontophoretic or galvanic current. The terms “iontophoresis” and “iontophoretic” as used herein refer to the introduction or transport of a substance such as a medicament into or through a skin, tissue, or other biological surface by the application of an electric current or electromotive force. According to one embodiment, the substance to be transported is completely charged, completely uncharged, or partly charged and partly uncharged. Alternatively, more than one substance can be transported, in which all of the substances are completely charged, completely uncharged, partially charged, or some of the substances are at least partially charged and some are uncharged.

According to one aspect, without being limited by theory, the transport of the substance is accomplished via electromigration, electroosmosis, or some combination of the two. It is understood that “electroosmosis” has also been referred to as electrohydroconesis, electro-convection, and electrically-induced osmosis. In one embodiment, electroosmosis of a substance into a tissue results from the migration of a solvent containing the substance as a result of the application of electromotive force to the substance.

The substance, in one embodiment, is a medicament. A “medicament” as used herein is any natural, homeopathic or synthesized material that may be applied to a biological substrate of choice. Thus, according to one embodiment, a medicament is a medicine, a healing or therapeutic substance, or a substance that promotes recovery from injury or ailment. “Medicament” may further be defined as a chemical or biological material that may be used or administered to a biological subject of choice (e.g. humans or animals) as an aid in diagnosis, treatment or prevention of disease or abnormal cosmetic condition, or relief of pain, or to control, diagnose or improve any physiological or pathological condition.

In a non-limiting example, the medicament may be comprised of a lotion applied to the skin of a human which contains a substance capable of imparting a beneficial effect. Alternatively, the medicament may take one of many forms and may be formulated, for example, as a liquid, gel, an ointment, a powder, a lotion, a foam, a solution, a cream, or any other known substance for application to a biological surface or tissue, depending on the nature of the medicament and procedure.

In one embodiment, the substance is a substance that effects or aids in the removal of cellulite. For example, one such substance is Galvanic Spa™ II Body Shaping Gel, which is sold by Nu Skin in Provo, UT. Alternatively, the substance can be any known substance that aids in the removal or reduction of cellulite. Alternatively, the substance is a substance that treats certain skin conditions or characteristics such as discoloration, lines or wrinkles, redness, irritation, oily skin, or acne. In a further alternative, the substance is a substance that aids in the exfoliation of the skin or increases skin brightness or smoothness. In yet another alternative, the substance is any substance that is capable of providing some beneficial effect via application to the skin. In one example, the substance contains CoQ10 and/or other colorless carotenoids, including those formulations or substances disclosed in U.S. application Ser. No.11/243,500, filed on Oct. 4, 2005, which is hereby incorporated herein by reference in its entirety. In another example, the substance contains an ingredient that inhibits arNOX, a free radical producing enzyme, including those formulations or substances disclosed in U.S. application Ser. No. 11/049,585, filed on Feb. 2, 2005, and U.S. application Ser. No. 11/478,210, filed on Jun. 29, 2006, both of which are hereby incorporated herein by reference in their entirety.

According to one implementation, a method or system is provided that relates to applying electrical current to a biological tissue or surface prior to, during (or at the same time as), or after application of the substance to be transported such that the substance continues to be absorbed by, transported into, or otherwise taken up by, the tissue or surface after the application of electrical current has ceased. In this embodiment, the electrical current enhances the beneficial effects of the substance, including, in some implementations, enhancing the beneficial effects of the substance after the electrical current is no longer applied. According to one embodiment, the electrical current is galvanic current and the biological surface is the human skin. Without being limited by theory, it is believed that the application of galvanic current to the skin enhances the beneficial effects of the substance by altering the skin to be permeable to the substance or by increasing the permeability of the skin to the substance. Alternatively, applying the current increases the ability of the skin to absorb the substance.

For example, according to one embodiment, the enhanced substance transport, absorption, or intake into or through the tissue or surface created by the application of the electrical current is sustained for a period of time ranging from about instantaneously upon removal of the electrical current to about 48 hours. Alternatively, the enhanced effects of the electrical current are sustained for a period ranging from about 1 hour to about 24 hours. In a further alternative, the enhanced effects are sustained from about 3 hours to about 20 hours.

In one embodiment, the electrical current is applied to the biological surface or tissue for a period ranging from about 5 seconds to about 24 hours. Alternatively, the electrical current is applied for a period ranging from about 15 seconds to about 12 hours. In a further alternative, the current is applied for a period ranging from about 30 seconds to about 6 hours. In yet another alternative, the current is applied for a period ranging from about 1 minute to about 15 minutes. In another implementation, the current is applied for a period ranging from about 1 minute to about 3 minutes. According to another implementation, more than one application of electrical current is applied to the surface or tissue during a period ranging from about 5 seconds to about 24 hours or any other range as discussed herein.

One implementation includes the electrical current being applied in an amount ranging from about 0.125 milliamps (mAmp) to about 50 mAmp. Alternatively, the current is applied in an amount ranging from about 0.125 mAmp to about 5 mAmp. In another alternative, the current is applied in any amount that a human can withstand being applied to the skin.

Another system or method, according to one implementation, relates to applying an electrical current to a patient's tissue or surface, such as the skin, and also applying—either simultaneously, before, or after the current—a substance and allowing the substance to remain on the surface or tissue for some period of time after application. According to one embodiment, the substance remains on the tissue or surface for a period of about 48 hours. Alternatively, the substance remains for a period of about 24 hours. In a further alternative, the substance remains for about 12 hours. In yet another alternative, the substance remains for an amount of time ranging from about 5 minutes to about 12 hours. In one embodiment, regardless of the amount of time, allowing the substance to remain on the skin for a period of time after application of the electrical current enhances the beneficial effects of the substance. In a further alternative, the enhanced transport, absorption, or intake created by the electrical current as described above, in combination with allowing the substance to remain on the skin, enhances the beneficial effects of the substance.

According to another variation, the application of electrical current to the biological surface produces a “depot effect” for some period of time such that any substance applied to the surface is retained below the surface. According to one embodiment, the depot effect is sustained for a period of time ranging from about instantaneously upon removal of the electrical current to about 48 hours. Alternatively, the depot effect is sustained for a period ranging from about 1 hour to about 24 hours. In a further alternative, the depot effect is sustained from about 3 hours to about 20 hours.

According to one embodiment, the electrical current can be applied by any method. That is, any known device can be used to apply the electrical current. For example, any known medical or cosmetic electrical current application device can be used. As such, the device can be any configuration of components that can be used to apply an electrical current to a target surface or tissue. In one embodiment, the device is lightweight and portable. In another embodiment, the housing is configured to be easily manipulated by a single hand of a user. That is, the user can engage the instrument in one hand or a portion thereof and freely orient the instrument to engage the active electrode on the instrument driving the current against the target area. In one embodiment, a system or method of use of an electrical current application device is provided in which a user can use the device to apply an electrical current to the user's own skin or other biological surface or tissue. In various embodiments, the user can also apply a medicament or other substance at the same time as the electrical current or prior to or after the application of current.

In accordance with one implementation, the electrical current application device is a device as described in either of U.S. Pat. Nos. 5,514,167 or 6,119,038. Such a device is a galvanic instrument which comprises the ability to function at variable current settings and run for longer timing periods, options not available in previously-produced handheld galvanic instruments. In some embodiments, the galvanic instrument comprises detachable heads to produce an instrument which is comfortable for use for longer periods of time. As discussed above, certain embodiments of the methods discussed herein in which current is applied for a longer period of time than previously known result in increased transport or uptake of a substance into the skin for an extended period of time after removal of the electrical current. By altering the instrument's time circuit to allow longer timing cycles and making the detachable heads of different shapes, suited to multiple applications (e.g. hair conductor, body conductor, facial conductor), the comfort, efficiency and efficacy of the galvanic instrument is improved, allowing the instrument to be used for longer periods of time, thus, maximizing the persistent effect.

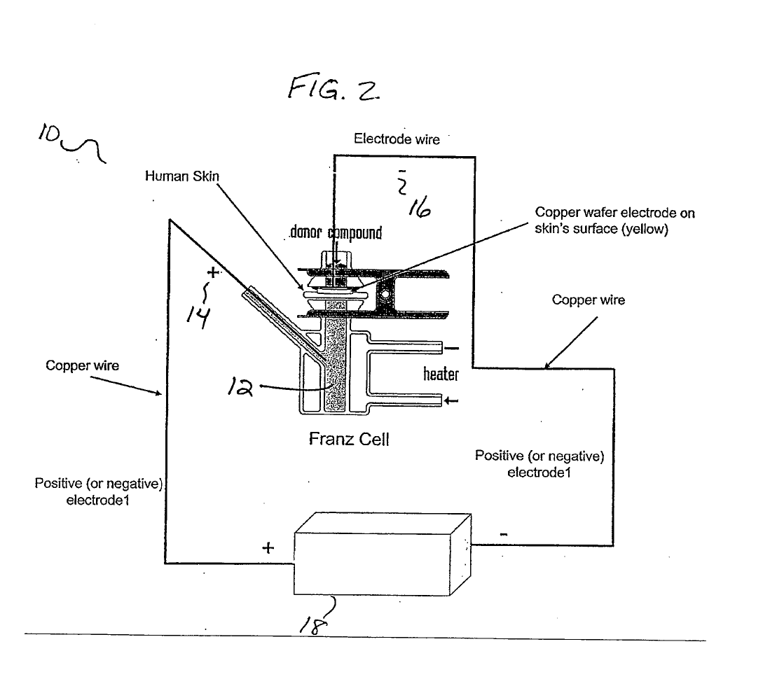

According to one embodiment, another method and apparatus relates to a modified Franz cell instrument and method of testing iontophoresis across a cellular membrane and the effects of an electrical current on the membrane after the electrical current has been removed. As shown in FIG. 2, a Franz cell device in accordance with one implementation of one embodiment includes a Franz cell configured such that an electrical current can be applied to the cellular membrane. In this embodiment, a electrical current application device 18 is operably coupled to the Franz cell via a copper wire that connects the two electrodes of the device 18 to the Franz cell. In this embodiment, the negative electrode 16 contacts the surface of the cellular membrane via a copper wafer electrode while the positive electrode 14 is placed in the lower Franz fluid reservoir 12 to complete the circuit. Alternatively, any configuration that allows for application of an electrical current to the cellular membrane placed in the Franz cell is contemplated. According to one embodiment, the apparatus as set forth in FIG. 2 can be used to test the effects of applying electrical current to a cellular membrane.

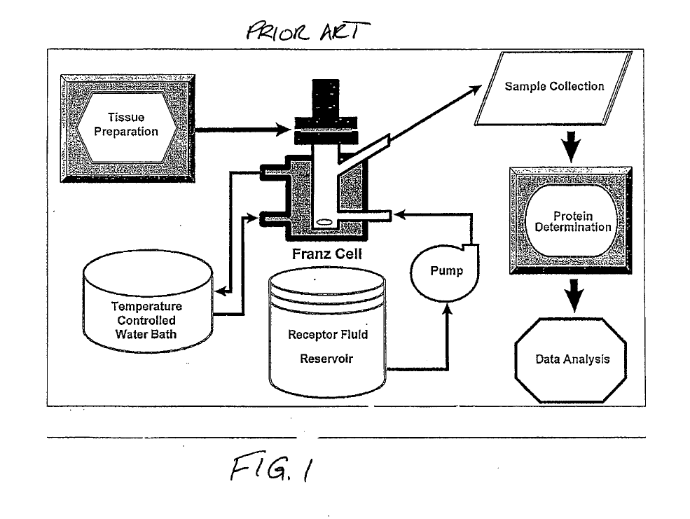

In comparison, a known Franz cell is shown in FIG. 1. See Hobson et al., Development and Use of a Quantitative Method to Evaluation the Action of Enzymatic Wound Debriding Agents Invitro, WOUNDS 10(4):106-110 (1998).

### EXAMPLE 1

The first example examines the effect of applying electrical current—using a galvanic device—on skin penetration by various medicaments/cosmetics.

Methods and Materials

To perform this examination, three modified Franz cells—each similar to the apparatus depicted in FIG. 2—were placed in series. The current through each skin in each Franz cell was measured and it was found that it was constant among all three units at 120 microamps, which is the current typically provided to the skin after the galvanic current application device 18 goes through its “Analyze” cycle. As is understood in the art, 1 milliamp equals 1,000 microamps. It was noted that if more than 3 Franz cell units were connected to the galvanic instrument 18, the current dropped. Thus, the replicate number for each group was limited to triplicates.

To determine the effectiveness of the current application device in enhancing the penetration of caffeine through human skin, a 1% caffeine solution was prepared in saline solution at a pH of 7.2 and an infinite dose (500 micro liters) applied to the surface of human skin mounted onto Franz cell units. One set of skin samples was left untreated to serve as the control group for passive skin penetration of caffeine. A second set was treated with one 3 minute cycle of the Galvanic unit delivering 120 microamps per skin. The standard convention for expressing the current “dose” the skin receives is Current×Time. Thus, the skin in this group received 360 microamp-minutes of current (120 microamps×3 minutes). A third group of skins were treated with 3 cycles of current (1080 microamp-minutes [shown as 1.1 mA]), and the fourth group received a dose of 4.32 mAmp-minutes (12 cycles at 3 minutes/cycle delivering 120 microamps).

Results

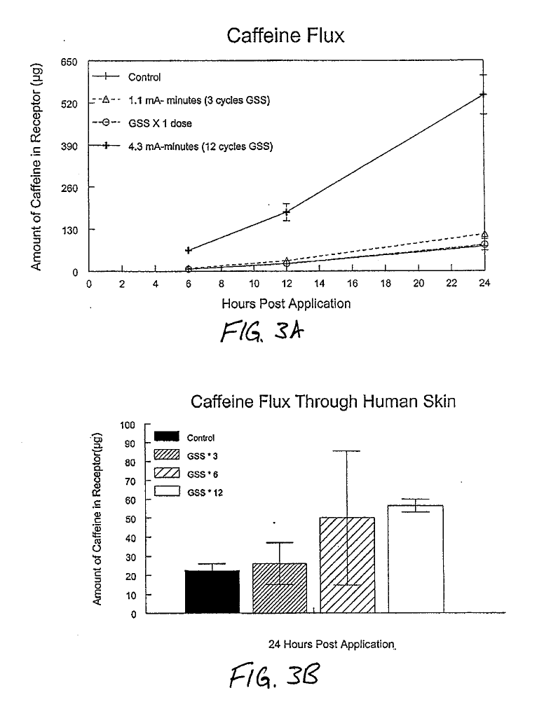

FIG. 3A shows the results of this experiment. Samples were collected at 6, 12 and 24 hours and the amount of caffeine present in the receptor fluid determined by HPLC. As can be seen a dose of 4.32 mAmps-min (12 cycles) produced an increase in skin penetration that was detectable as early as 6 hours. At 24 hours the amount of caffeine that had penetrated skin samples treated for 12 cycles was 6 times that of the control group. In contrast, a single “dose” of 1 cycle of the galvanic device was ineffective in causing any detectable penetration enhancement of caffeine through the skin. Skin samples treated for 3 cycles showed some marginal enhancement of penetration of caffeine at 24 hours compared to control. Accordingly, skin samples treated multiple times over an extended period of time experienced significant benefit.

Follow-Up Experiment Applying 6 Cycle Dose

Since a 12 cycle dose was very effective in enhancing skin penetration of caffeine while a 3 cycle dose appeared to be marginally effective, we repeated this study to determine the efficacy of a 6 cycle dose. The results of this study are shown in FIG. 3B. As can be seen, a 6 cycle dose corresponding to 2.2 mAmp-minutes did enhance delivery of caffeine through human skin. Note that again in this experiment the 3 cycle dose appears to cause some small enhancement in skin penetration.

### EXAMPLE 2

Further experiments were performed to determine the optimum current and time conditions to achieve enhanced penetration of a given compound into the skin.

Methods and Materials

For these studies, caffeine again was the test compound, and a different iontophoretic instrument was used which: 1) allows for the use of a high number of replicates (using 5 Franz units per experimental condition; 2) displays the current levels going through the skin in real time; and 3) allows the operator to pre-select a given dose (current-time) which the unit will produce and then automatically shut down at the end of the dose.

For the first studies, Franz cells were dosed once with either: 1) no current (control), 2) 2 mAmp-minutes, 3) 5 mAmp-minutes, or 4)10 mAmp-minutes. Samples of the Franz receptor fluid were analyzed at 6, 12 and 24 hours after the current dose.

Results

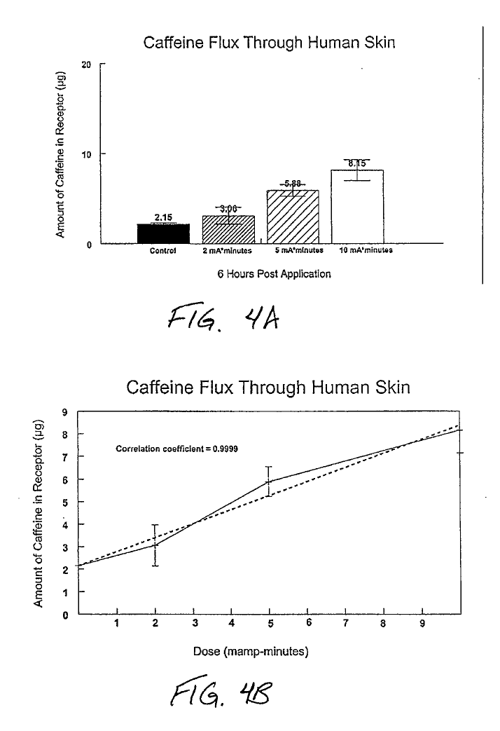

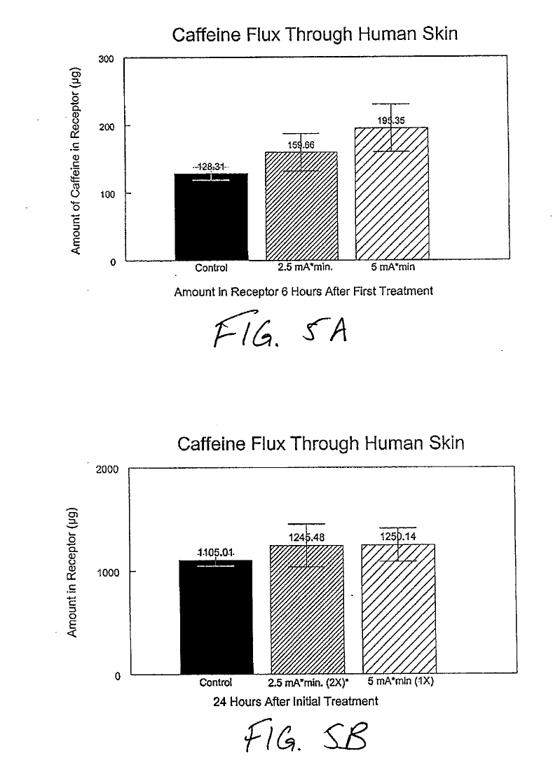

The results from the 6 hour time point are shown in FIGS. 4A and 4B. In this study, a 2 mAmp-minute dose produced a 1.5 fold increase in the penetration of caffeine as early as 6 hours after treatment. A 5 mAmp-minute dose produced a 3 fold increase and a 10 mAmp-minute dose a 4 fold increase in the amount of caffeine penetrating the skin. When a line graph of the data was analyzed by linear regression, the curve shown in FIG. 4B was obtained. From this data, one can predict that a current dose of as little as 1 mAmp-minute will provide skin penetration enhancement. A 5 mAmp-minute dose can be expected to produce a large (3 fold) enhancement in the penetration of caffeine through the skin.

### EXAMPLE 3

The next experiment related to the possibility that 2 smaller “doses” separated by several hours would achieve the same skin penetration “enhancement” benefit as a single larger dose.

Methods and Materials

Since the data above demonstrated clearly that a single 2.5 mAmp-minute dose provides a penetration enhancement which is about 50% that achieved by a single 5 mAmp-minute dose, we used these dosing parameters to conduct this multi-dosing study. Because a consumer might use this product morning and evening, we separated the dose treatments by 10 hours. The experimental protocol involved applying a saline solution of 1% caffeine (pH 7.2) to human skins mounted in Franz cells. The groups were: 1) control-no dosing, 2) a dose of 2.5 mAmp-minute (0.375 mAmps for 6.67 minutes) repeated at 10 hours, and 3) a single 5 mAmp-minute. All groups contained 5 replicates. For these studies, the caffeine was only applied to the skin during the dosing phase. When a second current application was applied to the 2.5 mAmp-minute group at 10 hours, not only was the caffeine solution added to these skin samples, but it was also placed on the 5 mAmp-minute skins even though these were not treated again with current. This controlled for any skin penetration simply due to the presence of the caffeine solution on the skin. After dosing was completed any remaining caffeine solution was removed from the skin.

Because of the long duration of these studies, dosing was usually initiated at 7:00 am and 6 hours later the first samples were collected for analysis. At T=10 hours (5 pm) a second sample was collected and the 2.5 mAmp-minute group dosed a second time. At T=16 hours (11:00 pm) another sample was collected from the lower reservoir, and at T=24 the final samples were collected.

Results

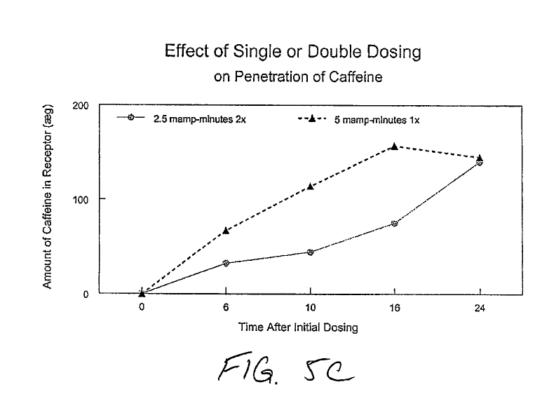

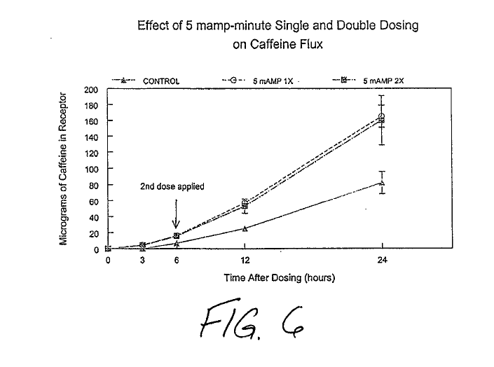

As can be seen in FIGS. 5A and 5B, the initial dosing resulted in penetration enhancement which was detectable as early as 6 hours post-dosing for both the 2.5 mAmp-minute group and the 5 mAmp-minute group with enhancement being higher, as expected, for the 5 mAmp-minute group. Interestingly, however, by 24 hours, there was no significant difference between the amount of caffeine that had penetrated the skin from the 2.5 mAmp-minute group that had been dosed twice and 5 mAmp group that had been dosed only once. The line graph in FIG. 5C displays the penetration of caffeine for all the time point with the amount of caffeine from the control group penetrating the skin subtracted from each value. From this data, it appears that although a 5 mAmp-minute dose provides a more rapid flux of caffeine through skin, dosing twice with 2.5 mAmp-minutes results in an equal amount of skin penetration over the long term (24 hours).

Discussion

Without being limited by theory, there are various possible interpretations of the data presented above and which is correct depends on how current acts on the skin and on the compound being tested. Two such possibilities are: 1) current alters the permeability properties of the skin itself creating “pores” that allow the compound to move through or 2) the permeability properties of the skin remain unchanged and the current simply provides a driving force behind the compound thus “forcing” it through a semi-permeable barrier. If possibility 1 is correct, then either: A) the current produces an increase in skin permeability that is immediately lost when current is removed, or B) the charge produces an increase in skin permeability that returns to its base level of permeability slowly after current is removed.

The results set forth in this example along with Examples 1 and 2 confirm that the skin experiences a prolonged beneficial effect which lasts for several hours after the termination of current. Possibility 1B, which states that the current alters the permeability properties of the skin and that this increased permeability only slowly returns to baseline after the removal of current (thus skin permeability remains elevated for a while even after current is removed), cannot be answered by this experiment because we removed the remaining caffeine from the skin immediately after the dose. If the skin remained “permeable enhanced” even after the current is removed, we would detect this only if we left the caffeine solution on the skin after the removal of current. Any caffeine on the skin after dosing would continue to move through the skin at an accelerated rate because of the continued increased skin permeability. Additional experiments discussed below have helped to address this possibility.

Regardless of the mechanism of current enhanced penetration the results suggest that repeated dosing for short times can result in nearly the same skin penetration enhancement as single dosing for longer times. Given that a more even and continuous penetration of any given compound typically achieves better bioactivity at the target site (in this case adipose cells) it seems probable that repeated dosing with lower currents might produce higher efficacy than would be achieved by use of a single large dose

### EXAMPLE 4

As is shown in 5A, 5B, and 5C, similar enhancement of skin penetration of caffeine can be achieved by a single dose of 5 mAmp-minutes or two doses of 2.5 mAmp-minutes. The next question is whether or not this relationship continues to hold as the current level applied to the skin is increased. That is, for a finite amount of caffeine placed on the skin, is there a current “dose” which causes a maximum penetration of caffeine that cannot be further enhanced by repeated dosing?

Methods and Materials

Since the data with a 5 mAmp-minute dose showed a large enhancement of caffeine penetration, we conducted studies to look at the difference between a single 5 mAmp-minute dose and two 5 mAmp-minute doses. For this study, the caffeine solution was not removed from skins immediately after dosing but left on the skin for the duration of the experiment.

Results

The results from this study are shown in FIG. 6. There are two interesting observations from this experiment. First, enhanced penetration of caffeine can be detected as early as 3 hours after dosing which suggests that caffeine penetrates the skin quickly and will reach the dermis and below (the site of adipose tissue) within a couple hours of treatment. The second observation is that, at least in this study, a single 5 mAmp-minute dose appears to provide a near-maximal enhancement of skin penetration of caffeine since repeating this dose 6 hours later causes no additional accumulation of caffeine in the receptor at 24 hours.

Discussion

One unlikely interpretation of this data, without being limited by theory, is that a single 5 mAmp-minute dose drives all the caffeine from the amount on the skin's surface that is capable of being “driven”. Thus, since for these studies no additional caffeine is applied to the skin's surface prior to a second dose, if all the caffeine that is capable of moving through the skin has in fact passed the stratum corneum during the first 5 mAmpminute dose, a second dose will be ineffective. This possibility seems unlikely since the total of amount of caffeine present in the receptor at 24 hours is only a fraction of that applied to the skin (4% of applied dose) and thus, there is still a large amount of caffeine that should be available to be “iontophoresed” during the second pulse. Again without being limited by theory, a more likely interpretation of the data is that a 5 mAmp-minute dose increases the permeability of the skin to a level that does not allow the skin to return to its original non-permeable state. Thus, the skin stays permeable for many hours and continues to allow enhanced penetration of caffeine (possibility 1B from above).

### EXAMPLE 5

Although our previous studies showed that two 2.5 mAmp-minute doses provided approximately the same level of skin penetration of caffeine as a single 5 mAmp-minute dose, one experiment not yet performed was a comparison of the caffeine flux from a single 2.5 mA dose with the penetration level seen with the use of two 2.5 mA doses.

Results

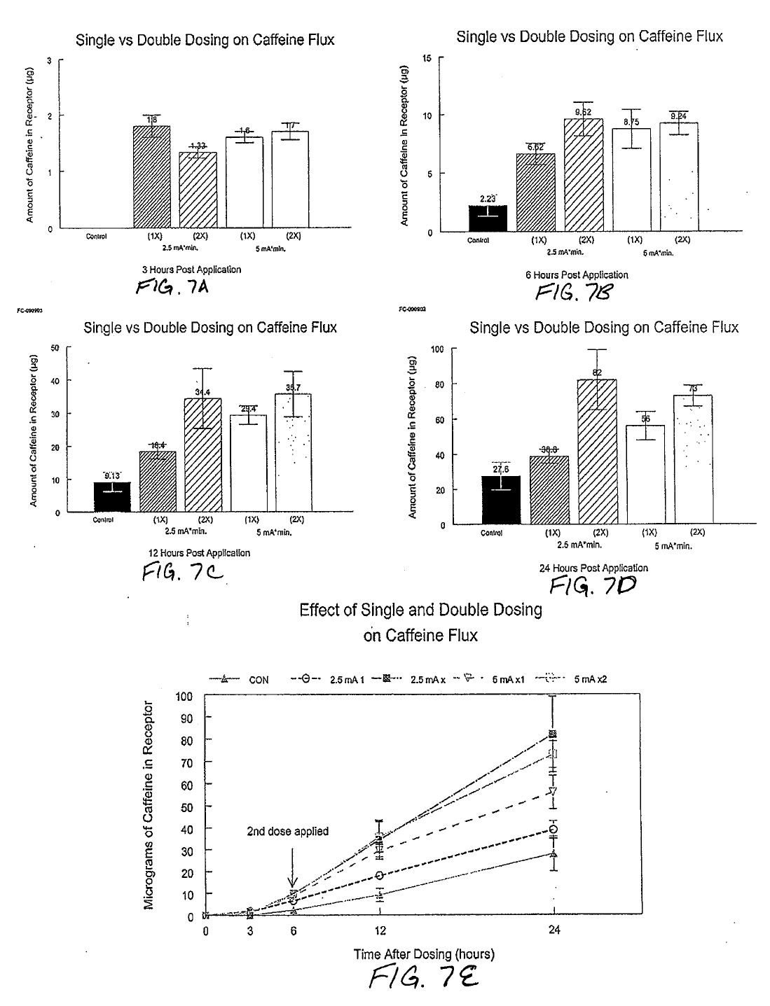

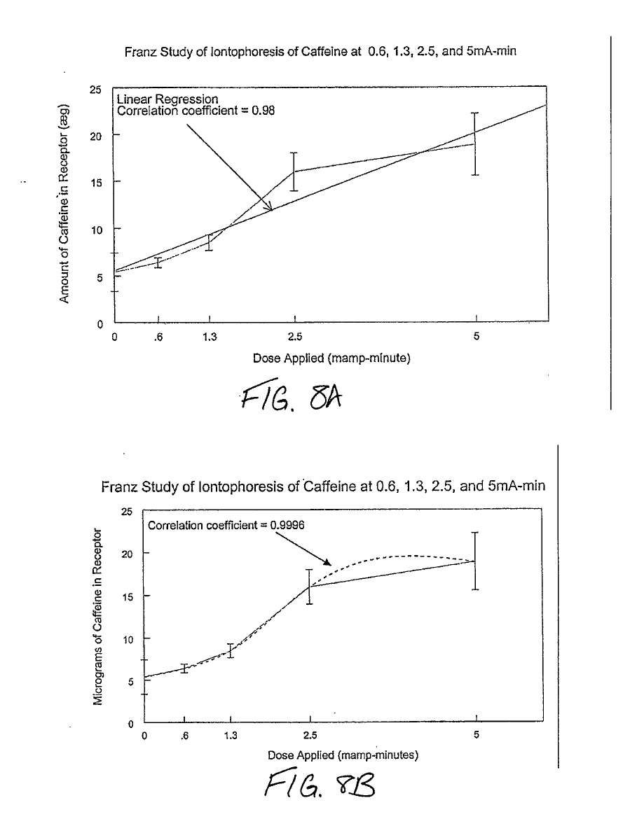

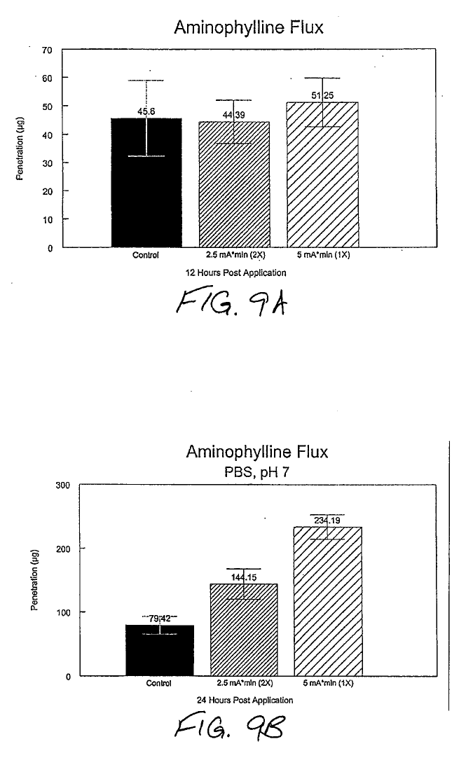

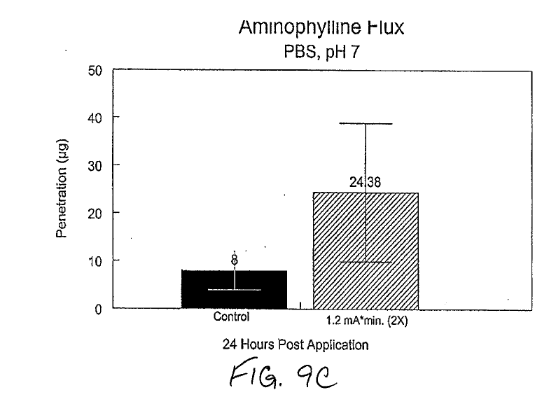

The results of this study are shown in FIGS. 7A through 7E. The results of this study show that: a single 2.5 mAmp-minute dose produces skin penetration enhancement that can be detected as soon as 3 hours after dosing (note the control skins show no presence of caffeine in the receptor at 3 hours); two 2.5 mAmp-minute doses produce significantly more skin penetration of caffeine than one dose does; at 24 hours two 2.5 mAmp-minute doses produce as much caffeine penetration as a single 5 mAmp-minute dose (which we've seen before); and a double 5 mAmp-minute dose does not appear to significantly enhance caffeine skin penetration much more than that seen with one 5 mAmp-minute dose (as we saw previously).

### EXAMPLE 6

Although the above experiments have examined various effects of either a 2.5 or 5 mAmp-minute dose on penetration enhancement of caffeine, in terms of the application of the Galvanic device, a question to address is how much application time is needed for this device to provide some level of skin penetration enhancement for any of the 5 compounds being tested.

Methods and Materials

We conducted dose-response studies to begin to answer this question. For these studies, skins were dosed only once with either 0.6, 1.3, 2.5 or 5 mAmp-minutes with the current held constant at 375 uamps, the maximum output of the Galvanic unit. To each skin 400 microliters of a 1% caffeine in saline solution was applied. In order to minimize evaporation during the study, the caffeine solution was gelled slightly with hydroxyethylcellulose. Six hours after dosing, samples from the lower receptor fluid were collected and analyzed for caffeine.

Results

The results are shown in FIGS. 8A and 8B. Although the linear regression analysis in FIG. 8A shows a correlation coefficient of 0.98, we also conducted a correlation of the data using a sigmoidal curve fit program. As shown in FIG. 8B, the data conforms nicely to a sigmoidal distribution with a correlation coefficient of 0.999.

Discussion

The results from the above study shows that a single dose of 0.6 mAmp-minutes will enhance skin penetration of caffeine; and the fact that the data conforms more accurately to a sigmoidal rather than a linear curve suggests that above a certain current level (between 3-5 mAmp-minutes) maximal skin penetration occurs and the use of a higher current will not further enhance skin penetration.

### EXAMPLE 7

Because the conditions for evaluating the effect of current on skin penetration have been established for caffeine, work to evaluate the other 4 compounds was confined to determining what current, if any, could alter skin penetration of either theobromine, aminophylline, thiotane or ethyl nicotinate.

Methods and Materials

Solutions of all compounds (1%) were prepared using buffered saline with the exception of theobromine which is virtually insoluble in any solvent except dilute alkali. Thus, for a 1% theobromine solution, a Tris-glycine buffer at pH 12 was chosen. The pKa values for ethyl nicotinate, aminophylline, and theobromine are 3.35, 5.0 and 9.9 respectively. Thus, at pH 8.0 ethyl nicotinate and aminophylline can be expected to be negatively charged. By putting theobromine into 0.1N NaOH at pH 12, this compound is ionized to a negative charge. No information was readily available regarding the pKa of Ergothioneine (Thiotaine) but based on its structure, it would appear to have a relatively low pKa and thus be negatively charged at pH 8.0.

For experiments, the electrodes from the Phoresor were applied to the Franz unit with the negative electrode in contact with the skin's surface and in contact with the compound solution being applied. Each compound was tested on 5 skins for skin penetration without current and 5 skins for skin penetration with current. Since a 5 mAmp pulse had been shown previously to enhance delivery of caffeine, this current was chosen.

Note that the time=0 for the bar graphs discussed below represents the time at which the galvanic current was terminated.

As can be seen in the figures below, the skin penetration of all compounds was enhanced by applying a current to the surface of the skin.

Aminophylline

A 60 microliter solution of aminophylline in PBS (pH 7.0) gelled slightly with 0.5% HEC was applied to human breast skin mounted in Franz cell chambers. A 0.4 mAmp current was applied for either 6.25 minutes (for a 2.5 mAmp×time dose) or 12.5 minutes to produce a 5 mAmp×time dose. At 6 hours samples were withdrawn for analysis and a second dose applied only to the skin receiving a 2.5 mAmp×time pulse at T=0. Samples were withdrawn for analysis at T=12 hours and again at T=24 hours.

As can be seen in FIG. 9A, at 12 hours only the 5 mAmp dose produced enhanced skin penetration. Note also the relatively high skin penetration of aminophylline seen in control skin (no current applied). The relatively high permeability of aminophylline through skin has been reported elsewhere. The 12 hour data only a modest increase in skin flux of aminophylline produced by a 5 mAmp×time pulse, and the data at 24 hours clearly shows that either a double 2.5 mAmp×time pulse or a single 5 mAmp×time pulse will enhance skin penetration of aminophylilne. In this experiment, however, two treatments of 2.5 mAmp×time did not equal the skin penetration seen with a single 5 amp×time pulse.

We also looked at the effect of a lower current dose on skin penetration of aminophylline. In the study shown in FIG. 9B, two 1.2 mAmp×time doses were applied to skin with a 1% aminophylline solution. This low dose also proved to be effective in enhancing skin penetration.

We then looked at the ability of a single 2.5 mAmp×time dose to enhance aminophylline delivery into the skin and for this experiment we also increased the pH of the solvent to see if a higher pH might increase the dissociation of aminophylline into its anion form and thereby increase skin penetration. As shown in FIG. 9C, a single dose was found to be effective in increasing skin penetration approximately 1.7 fold over a 24 hour period.

By extrapolating the data from all of the aminophylline studies, it is possible to establish a crude estimate of efficacy at lower doses. Thus, a current dose (amp×time) of 1 mAmp should provide a 23% enhancement in skin penetration of aminophylline over control values. Even a 500 microamp dose should provide a 9% enhancement in skin penetration. This level of penetration enhancement is slightly less than that seen with caffeine (see above data for 2.5 mAmp pulsing) but this difference is likely due to the higher basal skin penetration rate of aminophylline.

From the above data one can estimate that a dose as small as 0.5 mAmp×time will still provide enhanced skin penetration of aminophylline. Given the Galvanic unit's current output of 0.12 mAmps (theoretical maximum 0.375 mAmps), and a typical human surface current of 15 microamps, to achieve a 0.5 mAmp dose, the consumer would have to apply current twice a day allowing 16 minutes for each treatment. Further, it appears that placing aminophylline in a buffered solution with a pH of 8 or greater will enhance the current induced skin penetration.

Theobromine

As mentioned above, theobromine is essentially insoluble in any solvent other than alkali. However, its pKa value is 9.9. Thus, its rate of dissociation is not going to be high in an aqueous solvent. in order to shift the equilibrium to the anion form of theobromine we prepared a 1% solution in Tris-glycine buffer adjusted to pH 12 with IN NaOH. Franz studies were then carried out using a 2.5 mAmp×time pulse. Since theobromine can exhibit both a positive and negative charge, we used both a positive and negative electrode on the skin surface.

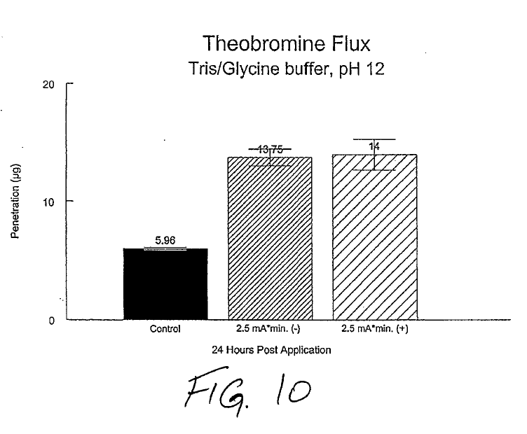

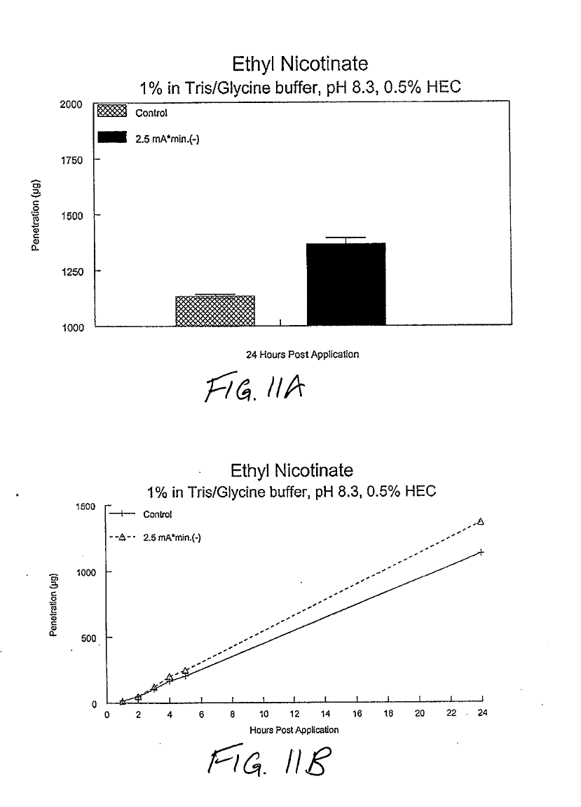

As shown in the graph of FIG. 10, the results showed that enhancement of skin penetration could be achieved with either polarity of electrode, and that the enhancement was pronounced. Note that the negative sign (−) in the figure indicates the polarity of the electrode at the surface of the skin. As is shown, a single 2.5 mAmp dose produced a greater than 2 fold increase in skin penetration.

It is worth noting that theobromine penetrates the skin relatively poorly from the solvent used. However, the 2-fold difference between a 2.5 mAmp×time current enhanced and non-enhanced is similar to that seen with caffeine and slightly higher than that seen for aminophylline. Thus, it seems likely that this compound, like the others, will show enhanced skin penetration even with current dosing as low as 0.5 mAmp×time.

Ethyl Nicotinate

As mentioned previously, ethyl nicotinate is extremely skin permeable even in water and thus, demonstrating enhanced skin penetration by the application of current is challenging. For example, if one looks at the amount of ethyl nicotinate penetrating human skin over 24 hours from a “control” group, the amount of compound in the receptor fluid is over 1000 micrograms which is over 10 times higher than even the highest values measured for either caffeine or aminophylline and about 100 times higher than the skin penetration rate seen for theobromine. However, even with this high level of skin permeability, a single 2.5 mAmp×time dose enhanced skin penetration.

Given the high skin penetration rate of ethyl nicotrate, we repeated the Franz study to determine how quickly ethyl nicotrate can penetrate skin to produce detectable amounts in the low receptor fluid. As can be seen in FIGS. 11A and 11B, the compound is detectable in the lower reservoir fluid as early as 2 hours after applying it to the surface of the skin. The graphs also show that the enhanced effects of a 2.5 mAmp dose is detectable about 4 hour after the addition of a 1% ethyl nicotinate solution to the skin's surface and continues to become more distinct from the control group by 12 and 24 hours. Thus, it appears that the enhancement effect of current will become more noticeable and more significant the longer the product is applied.

Thiotaine (Ergothioneine)

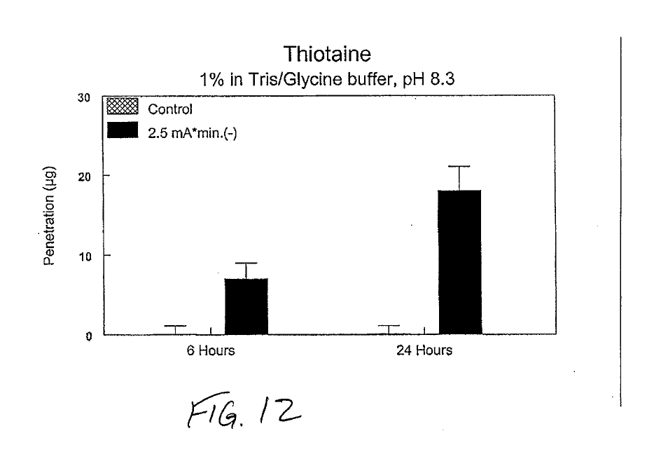

The results are shown in FIG. 12. As can be seen, unlike the other compounds, Thiotaine is very skin impermeable in the simple solvent system used in these studies. Even at 24 hours, no compound had penetrated the skin to reach the lower chamber reservoir. In contrast, the skin samples treated with a single dose of 2.5 mAmp×time showed measurable Thiotaine levels in the lower reservoir at both 12 and 24 hours. Thus, for this compound, if no skin penetration enhancers are used in the product formulation, the application of current will be critical for any of the compound to penetrate past the stratum corneum.

Discussion

Skin penetration for all the compounds studied was enhanced by applying a current to the skin. Detectable increases were seen using a current pulse as low as 0.6 mAmp×time while obvious enhancement was seen with currents above 2 mAmp×time.

The most permeable compound in this study was ethyl nicotinate while the least permeable compound was Thiotane. Caffeine, aminophylline and theobromine had somewhat similar skin penetration rates from a simple PBS buffer, although it appears that of these methylxanthines, skin penetration of theobromine is less.

For these studies simple buffer systems were used to solubilize the compound under investigation and no effort was made to use penetration enhancers to increase skin flux. By altering the pH of the medium the compound is placed in, it is likely that the efficacy of the Galvanic unit can be enhanced. Further, developing formulations that contain skin penetration enhancers (e.g. ethoxydiglycol) may markedly enhance skin penetration of these compounds with or without the use of current.

### EXAMPLE 8

The following experiment relates to the effectiveness of body shaping gel when used in conjunction with application of galvanic current.

Background

Excess adipose tissue, more commonly known as cellulite, is a condition about which relatively little is known. In medical literature, cellulite is known as adisposis adematosa, dermopanniculosis deformans, status protrusus cutis, and by several other medical terms. Cellulite can be located anywhere on the body that contains excess subcutaneous fat and appears as uneven, bumpy skin texture often seen with side lighting of the affected area. Skin influenced by cellulite has been described as having an “orange peel” or “cottage cheese” appearance. Obesity is not necessary for the presence of cellulite since the pattern of adipose deposits that lead to cellulite may be genetically determined. It is not commonly seen in men and certain areas are more likely than others. It is most commonly seen on the upper outer thighs, the posterior thighs (banana roll), and buttocks, but can also be seen on the breast and upper arms.

The cause of cellulite deposits is unknown. Although the word cellulite means “cell inflammation,” cellulite cannot be considered a disease. It is considered a normal body change associated with puberty and it is estimated that 85% of females are afflicted with the condition. It may be seen most often in women because the female subcutaneous fat is sequestered into discrete pockets by the presence of septa or separating membranes. The current theory holds that cellulite is an inflammatory process that results in breakdown of the collagen in the dermis leading to subcutaneous fat ruptures. The onset of cellulite with puberty has led some researchers to evaluate the effect of the elevated levels of collagenases and gelatineases that occurs during menstruation. Elevation of collagenases can cause the breakdown of the fibrillar collagens present in the dermis, and gelatinase elevation can lead to an influx of immune system cells that contribute to inflammation. With repeated cyclical collagenase production, more and more dermal collagen is destroyed, resulting in the worsening of the cellulite seen with age. When enough collagen is destroyed to weaken the dermis, it can rupture and allow fat to move between the structural fibrous septa found in female fat. If more fat is present, the rupture is larger. Because the dermal capillary network is damaged in the process, excess fluid is retained with the dermal and subcutaneous tissue further accentuating the appearance of cellulite. This loss of the capillary network is thought to be due to engorge fat cells clumping together and inhibiting venous return.

Xanthine derivatives from plants can be effective against cellulite because they are able to stimulate the fat breakdown in adipocytes. Theobroma cacao (cocoa) extract containing theobromine from cocoa is very effective. Another plant extract Chrysanthellum indicum can assist with increased fat breakdown (lipolysis) by linking the a2 adrenergic together to prevent the generation of cellular signals that turn off lipolysis (fat breakdown).

Capillary and lymphatic circulation improvement can also assist with the treatment of cellulite. An extract of Chrysanthellum indicum can reduce edema and inflammation and promote tissue drainage and the elimination of toxins.

Methods and Materials

Twenty-seven women between the ages of 25 and 50, with body weight within 30% of their body mass index, were enrolled in an 8-week clinical study to demonstrate product efficacy. Clinical investigator assessments (investigator were blinded to the treatment), subject assessments, and thigh measurements were conducted. The treatment regimen consisted of the use of Galvanic Spa™ II Body Shaping Gel applied to the test area twice per day, two times per week, and massaged into the skin using the Nu Skin® Galvanic Spa™ System II instrument. Both the Galvanic Spa™ II Body Shaping Gel and the instrument are sold by Nu Skin in Provo, UT. The instrument was configured to deliver a negative charge and set for 5 minutes.

Results

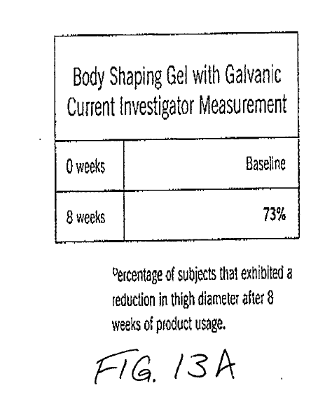

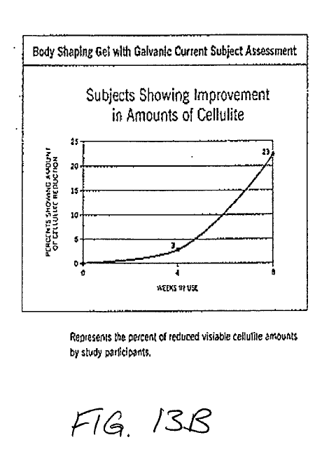

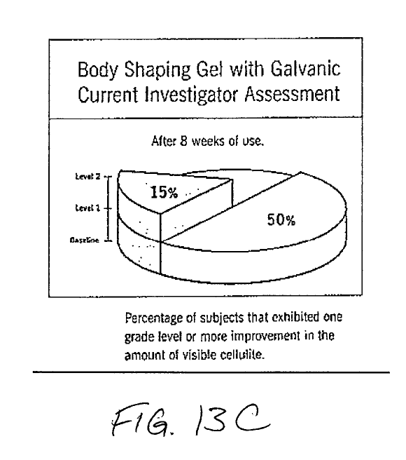

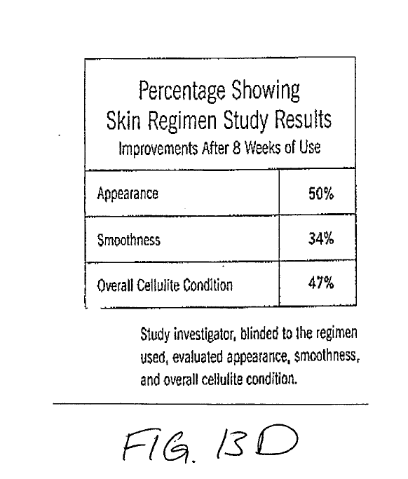

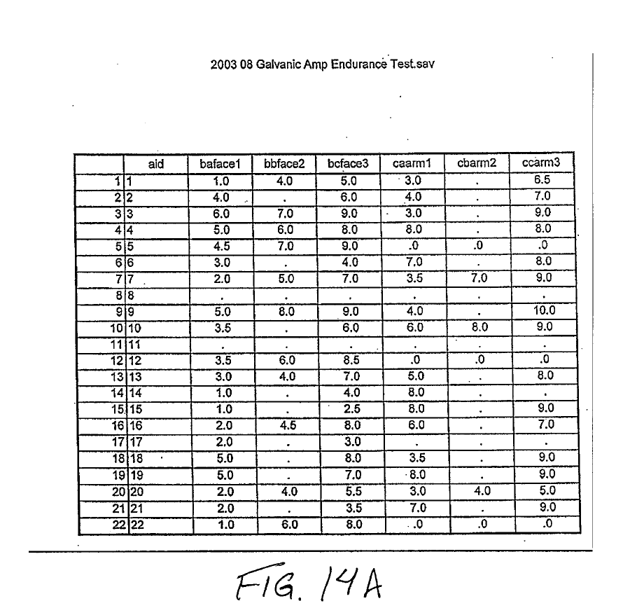

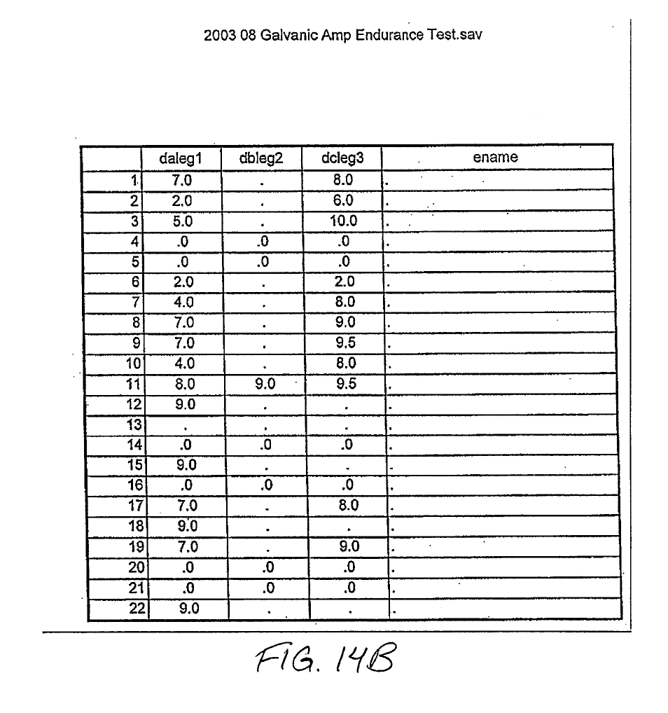

The circumference of the thigh of each subject was measured at 0 (baseline), 4, and 8 weeks by a professional grader and the number of study subjects demonstrating a reduction in thigh diameter was counted. Compared to baseline, 73% of subjects exhibited a reduction in thigh diameter at 8 weeks (p=0.005), as shown in FIG. 13A.

Study subjects evaluated the amount of cellulite visible at 0 (baseline), 4, and 8 weeks. The results at 8 weeks (compared to baseline), showed Galvanic Spa™ II Body Shaping Gel, when used with Galvanic Spa™ System II instrument, providing a controlled galvanic current, significantly reduced cellulite (p=0.001). Subjects documented a 23% improvement in the amount of cellulite, as shown in FIG. 13B.

Study subjects evaluated the amount of cellulite at 0 (baseline) and 8 weeks using a grading scale where 0=none, 1=minimum, 2=mild, 3=moderate, and 4=severe. Compared to baseline, at 8 weeks 50% of participants noticed more than 1 grade level improvement and 15% of participants noticed an improvement of more than 2 grade levels (p=0.001), as shown in FIG. 13C.

The study investigator, blinded to the regimen used, evaluated the appearance, smoothness, and overall cellulite condition of the test areas at 0 (baseline) and 8 weeks. At 8 weeks, improvements were seen in all parameters, as shown in FIG. 13D.

### EXAMPLE 9

In order to verify whether a higher electrical current would be suitable for human administration, research was done to find out what electrical current level consumers can tolerate at different parts of body.

Methods and Materials

This was a 18-subject non-blinded study recording LED levels when subjects first noticed any sensation and/or felt discomfort on one facial cheek, rear upper arm and outer upper thigh by using the Galvanic SpaTM System II instrument. By recording the LED levels and the description of sensations reported by each subject, sensible and intolerable LED levels can be determined.

Each LED level was then translated into voltage and current. LED (Light-Emitting Diode) was an electronic display when voltage was applied to the instrument. The actual current the 18 subjects first noticed and then felt uncomfortable can be determined.

In order to correlate the current applied to subjects in the spa and the current used in the instrument, the same 18 subjects applied an instrument at two pre-set settings and investigators recorded the sensations at each setting.

By noting the LED levels when each of the 18 subjects first noticed a sensation and later uncomfortable sensation and the actual current flow through at each LED level, the exact current that first caused a sensation and uncomfortable sensation with the instrument can be determined. When comparing the sensations the 18 subjects experienced with the instrument, a current that created the same sensation can be estimated in ratio.

Part A—Sensations on Cheek, Arm and Thigh with the Instrument

Spa estheticians cleansed areas on facial cheek, rear upper arm and outer upper thigh of each subject to remove oil and any undesirable impurities and then applied sufficient amount of Nu Skin® Galvanic Spa Pre-Treat Gel on the areas. Each subject held a moist electrode in his/her hand to complete the circuit while the esthetician applied another electrode roller to the test areas.

The instrument was set to deliver a negatively charged current to the electrode roller being applied to the test areas. The electrode roller was applied to areas on the facial cheek first, arm next and thigh at last. Investigators recorded the LED levels when subjects first described a sensation and then the point at the discomfort level.

Part B—Current Levels of the Existing Portable Instrument

Six instruments were tested to obtain the exact amount of current and voltage at two pre-set settings, 1-beep and 3-beep settings, to make a correlation with the current and voltage used in the instrument.

Part C—Sensations on the Facial Cheek with the Existing Instrument

The same 18 subjects applied the Nu Skin®) Galvanic Spa Pre-Treat Gel on their face and put the instrument on their face for skin analysis. The instrument automatically analyzed the skin and beeped 1, 2, or 3 times to indicate its setting according to the resistance of the skin. Subjects then moved the instrument slowly on their face and described any sensation to investigators.

If subjects did not receive the highest current setting (3-beep setting), a short circuit would be created across the flat chrome head and the instrument body. This set the instrument to the 3 beep or high current mode. Subjects then moved the instrument slowly on the face and described any sensation to investigators again.

Results

Part A—Sensations on Cheek, Arm and Thigh with the Instrument

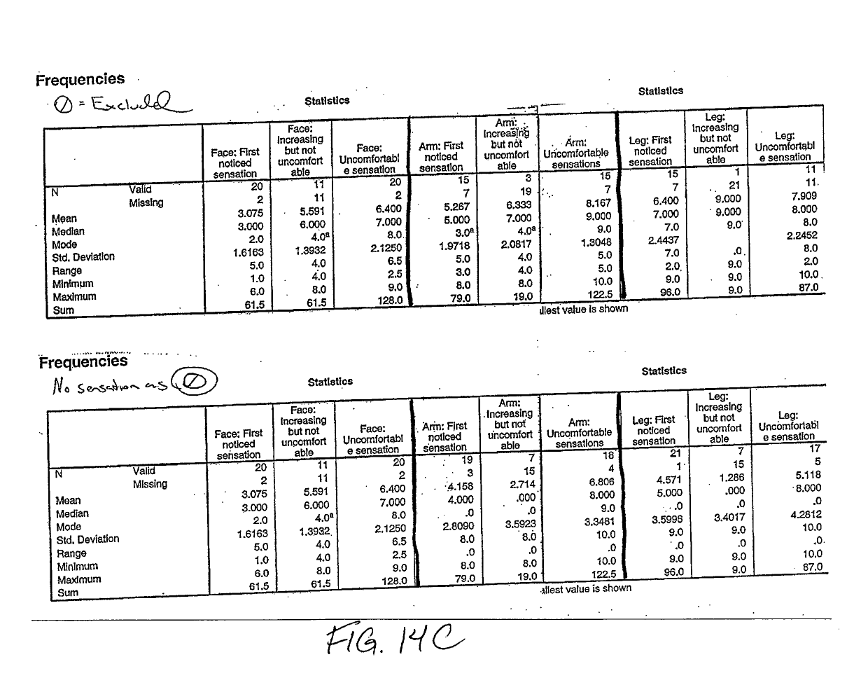

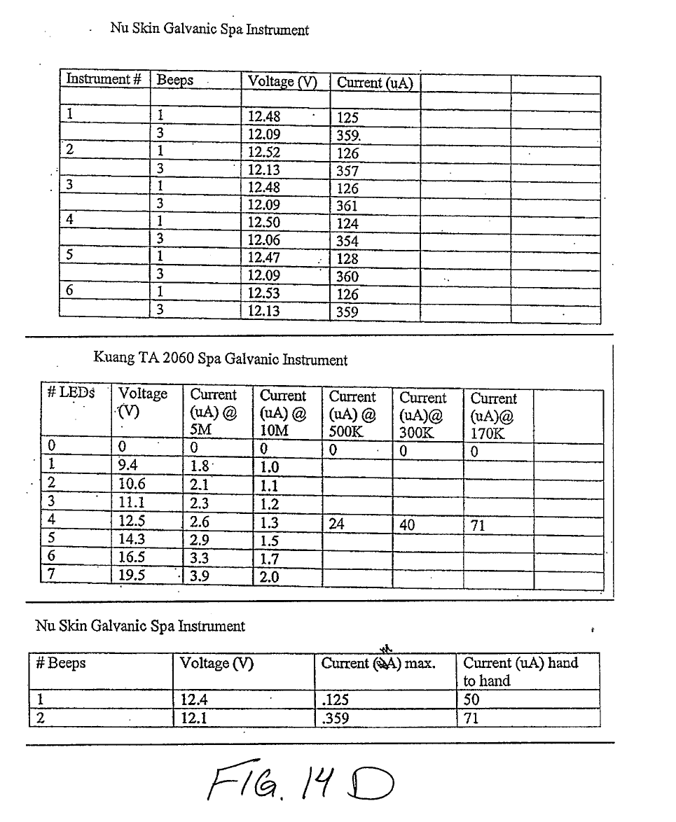

The results are shown in FIGS. 14A, 14B, and 14C.

Subjects noticed the first sensation on their facial cheek area when there was an average of 3.075 LED applied (approximately 11.205 vplts). On their rear upper arm areas, there was an average of 5.438 LED applied (approximately 15.264 volts). On their outer upper thigh areas, there was an average of 6.400 LED applied (approximately 17.700 volts).

Subjects notice an uncomfortable sensation on their facial cheek areas when there was an average of 6.4 LED applied (approximately 17.700 volts). On their rear upper arm areas, there was an average of 8.167 LED applied (approximately 26.137 volts). On their outer upper thigh areas, there was an average of 7.909 LED applied (approximately 23.681 volts).

Part B—Current Levels of the Portable Instrument

The results are shown in FIG. 14D. The instrument delivered an average of 12.50 volts (approximately 126 uA current) at 1-beep setting while an average of 12.10 volts (approximately 358 uA current) was delivered at the 3-beep setting.

Part C—Sensations on the Facial Cheek with the Instrument

100% subjects received 1-beep treatment (the lowest current). About 25% of the subjects had a sensation on the 1-beep setting (most of which are very slight sensation) while 75% of them had no sensation on the same setting. About 62.5% of the subjects had a sensation on the 3-beep setting (some of which are very slight sensation) while 37.5% of them had no sensation on the same setting.

Discussion

Facial cheek areas were the most delicate and sensitive areas when compared against the rear upper arm and outer upper thigh areas on the sensation of electric current. Whereas the rear upper arm areas were more to notice the first sensation than the outer upper thigh, the rear upper arm areas had a higher tolerance to an uncomfortable electric current level than the facial cheek area or the outer upper thigh area.

When the instrument delivered 12.50 volts at four LEDs, subjects first noticed a sensation on the facial cheek according to results shown in Part A.

The first noticed sensation on the outer upper thigh areas is at an average of 17.700 volts, which was the voltage level where the facial cheek areas were recorded as uncomfortable to intolerable.

The instrument delivered between 12.10-12.50 volts of current, which was slightly higher than the first noticed sensation on the facial cheek areas. When consumers used the instrument as directed, most received the 1 beep setting or 0.125 mA of current and were not likely to sense the current.

By evaluating the voltage level when the 18 subjects first noticed sensations and uncomfortable sensations and the voltage delivered by the instrument, it was determined that the instrument delivers an optimal level of current to consumers for the purpose of use on the facial areas.

In one embodiment, all settings can be auto-set by the instrument. The voltage levels required for the less sensitive areas (arms and legs) could be much higher than the level for the more sensitive areas (face).

### EXAMPLE 10

The following is a discussion of the possible compatibility of certain commercially-available products with certain embodiments of the methods and systems disclosed and claimed herein, using an electrical current application instrument such as the Nu Skin® Galvanic Spa™ System II instrument or the Phoresor® II Auto Model PM850 (available from IOMED, Inc., located in Salt Lake City, Utah) or an equivalent instrument. Some products that might be applied with an electrical current include: TruFace Line Corrector™, TruFace Essence™, TruFace Priming Solution™, TruFace Revealing Gel™, Clear Action Day Treatment™, 180 Cell Renewal Fluid™, and Celltrex CoQ10 Complete™. To that end, certain characteristics of each of these products is discussed herein as those characteristics may relate to the application of the product in conjunction with one or more embodiments of the methods and systems discussed herein.

It is understood that the following assessments are based on a review of published properties of the key ingredients in each product formulation as we understand the hypothetical interactions therein. No laboratory tests have been conducted to confirm these assumptions.

TF Line Corrector™

One component of this product is palmitoyl pentapeptide-3, which has a net positive charge at the specified pH of about 5.25 to 5.75. Thus, it is recommended that the “positive” or “treatment” setting on the electric current application device.

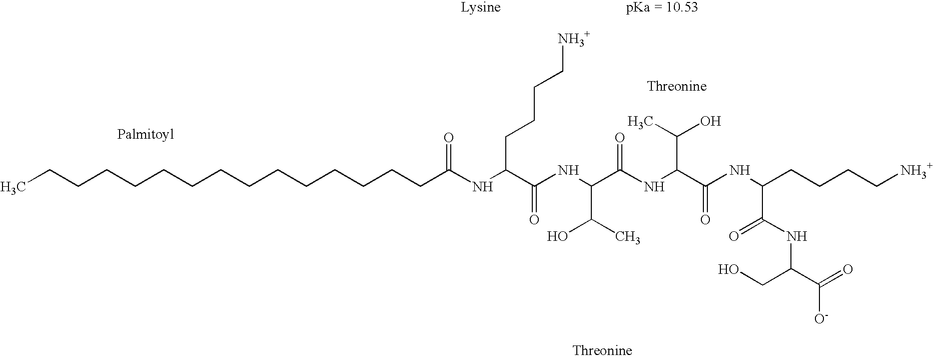

TF Revealing Gel™

One component of this product is gluconolactone, which has no net charge at the specified pH of 3.5.

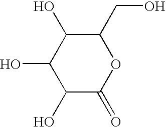

Another component of this product is lactobionic acid, which has a slight net negative charge at the specified pH.

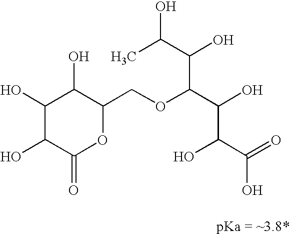

Another component of this product is arginine, which has a net positive charge at the specified pH.

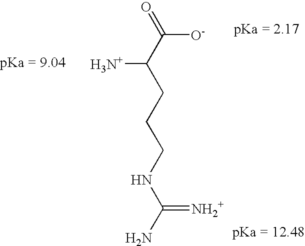

As noted above, gluconolactone has no significant charge, lactobionic acid has a slight negative charge, and arginine has a net positive charge. Thus, it may be best to use the galvanic instrument on the “positive” or “treatment” setting to facilitate the movement of the arginine into the skin by the iontophoretic mechanism and movement of gluconolactone and lactobionic acid via convective solvent (water) flow.

TF Essence™

This product is non-conductive and is unsuitable for use with a galvanic instrument.

TF Priming Solution™

One component of this product is DMEA, which has a net positive charge at the specified pH of 6.8.

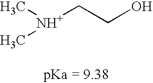

Another component of this product is citric acid, which has a net negative charge at the specified pH.

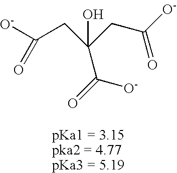

Another component of this product is an amino acid blend of lysine, glycine, proline, alanine, phenylalanine, leucine, valine, arginine, histidine, isoleucine, threonine, and citrulline.

Lysine, arginine, and histidine have a net positive charge at a pH of 7.

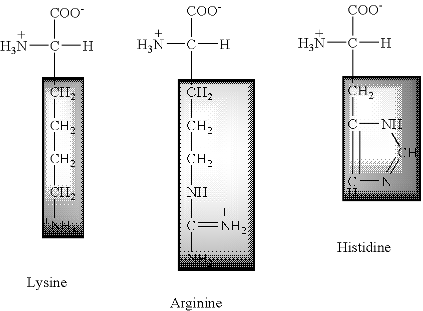

Citrulline, which is not shown, has a pKa of 9.4.

Phenylalanine, leucine, threonine, glycine, proline, alanine, isoleucine, and valine have no net charge at a pH of 7.

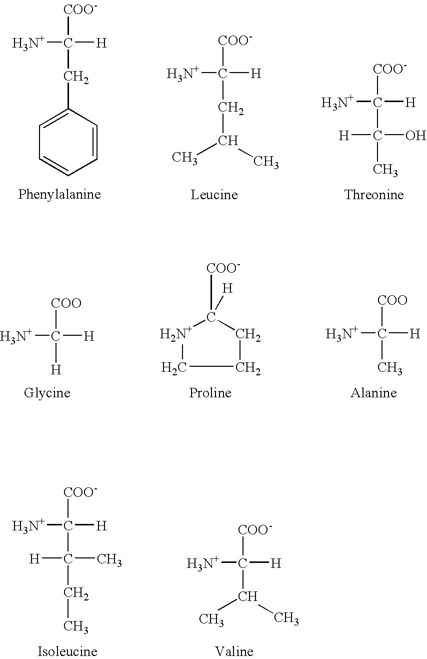

As discussed above, DMAE has a net positive charge in the product. Thus, it is recommended that the “positive” or “treatment” setting be used on the galvanic instrument. Other penetration-enhancing ingredients in the product as described above may be facilitated into the skin by the convective solvent flow (water) that occurs with the use of the instrument on the “positive” setting.

180 Cell Renewal Fluid™

One component of this product is gluconolactone, which has no net charge at the specified pH of 3.5 to 4.

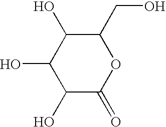

Another component is arginine, which has a net positive charge at the specified pH.

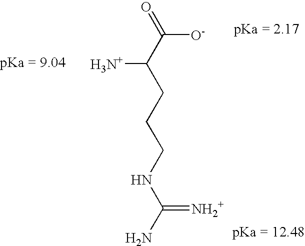

Another component is soy isoflavones (Genistein and Daidzen), which have no net charge at the specified pH.

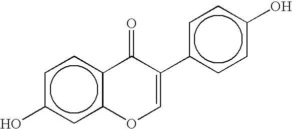

Another component is salicylic acid, which has a slight net negative charge at the specified pH.

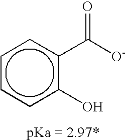

The characteristics of this product are somewhat similar to the TF Revealing Gel™. Thus, it may be best to follow the same recommendations set forth for that product.

Clear Action Day Treatment™

One component of this product is salicylic acid, which is set forth above and has a slight net negative charge at specified pH of 3.5 -4.0.

Another component is hexapeptide-2, which has a net positive charge at the specified pH of 3.5-4.0.

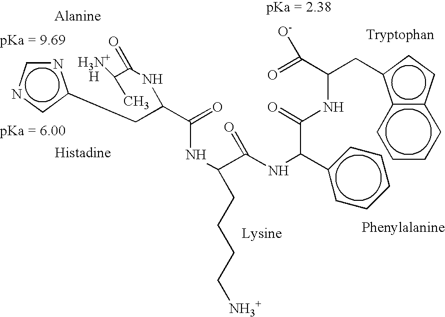

Another component is lactic acid, which has a slight net negative charge at the specified pH of 3.5-4.0.

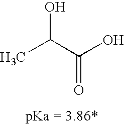

As discussed above, salicylic acid and lactic acid each have a net negative charge while hexapeptide-2 has a net positive charge. Thus, it may be best to use the galvanic instrument on the “negative” or “pre-treatment” setting. This may affect the initial delivery of the hexapeptide-2 into the skin. However, since the product is left on the skin, persisting changes in the skin following galvanic treatment may facilitate increased delivery of the hexapeptide-2 following application of the “negative” galvanic current.

Ceiltrex CoQ10 Complete™

One component of this product is ubiquinone (CoQ), which has no net charge at the specified pH of 5.2-5.8. More specifically, ubiquinone does not have a true charge, but the ketone oxygens likely have a partial positive charge (delocalization from resonance with the benzene ring and other oxygen) fundamental to its action as an antioxidant.

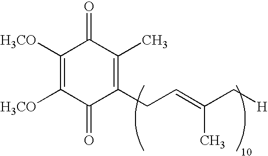

Another component is ascorbic acid (also referred to as “vitamin C”), which has a net negative charge at the specified pH of 5.2-5.8.

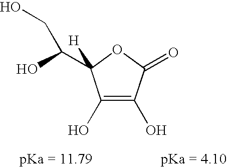

Another component is alpha-tocopherol (also referred to as “representative vitamin E”) which has no net charge at the specified pH.

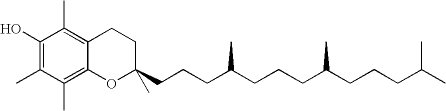

Although the present invention has been described with reference to preferred embodiments, persons skilled in the art will recognize that changes may be made in form and detail without departing from the spirit and scope of the invention.

What is claimed is:

1. A method of treating skin with an electrical current comprising:
applying a substance to the skin; and
applying an electrical current to the skin for a predetermined amount of time,
wherein transport or absorption of the substance into the skin is enhanced for a period of time after terminating the electrical current.

2. The method of claim 1, wherein the transport or absorption of the substance into the skin is enhanced for a period of time ranging from about instantaneously after terminating the electrical current to about 48 hours after terminating the electrical current.

3. The method of claim 1, wherein the transport or absorption of the substance into the skin is enhanced for a period of time ranging from about 5 minutes after terminating the electrical current to about 36 hours after terminating the electrical current.

4. The method of claim 1, wherein the transport or absorption of the substance into the skin is enhanced for a period of time ranging from about 10 minutes after terminating the electrical current to about 24 hours after terminating the electrical current.

5. The method of claim 1, wherein the transport or absorption of the substance into the skin is enhanced for a period of time ranging from about 30 minutes after terminating the electrical current to about 24 hours after terminating the electrical current.

6. The method of claim 1, wherein the transport or absorption of the substance into the skin is enhanced for a period of time ranging from about 1 hour after terminating the electrical current to about 24 hours after terminating the electrical current.

7. The method of claim 1, wherein the transport or absorption of the substance into the skin is enhanced for a period of time ranging from about 3 hours after terminating the electrical current to about 24 hours after terminating the electrical current.

8. The method of claim 1, wherein the substance is applied before the electrical current.

9. The method of claim 1, wherein the electrical current is applied before the substance.

10. The method of claim 1, wherein the substance and electrical current are applied at substantially the same time.

11. The method of claim 1, wherein the substance is a medicament.

12. The method of claim 1, further comprising allowing the substance to remain in contact with the skin for a predetermined period of time after terminating the electrical current.

13. The method of claim 1, wherein the electrical current is a direct current.

14. The method of claim 1, wherein a beneficial effect of the substance is enhanced.

15. A method of treating a biological surface with an electrical current comprising:
applying a substance to the surface;
applying an electrical current to the surface for a predetermined amount of time; and
allowing the substance to remain in contact with the surface for a predetermined period of time after terminating the electrical current,
wherein transport or absorption of the substance into the surface is enhanced for a period of time after terminating the electrical current.

16. The method of claim 15, wherein the transport or absorption of the substance into the skin is enhanced for a period of time ranging from about instantaneously after terminating the electrical current to about 48 hours after terminating the electrical current.

17. The method of claim 15, wherein the transport or absorption of the substance into the skin is enhanced for a period of time ranging from about 5 minutes after terminating the electrical current to about 24 hours after terminating the electrical current.

18. The method of claim 15, wherein the biological surface is skin.

19. The method of claim 15, wherein the substance is a medicament.

20. A device for testing application of electrical current to a cellular membrane, the device comprising:
(a) a Franz cell;
(b) a positive electrode operably coupled to the Franz cell;
(c) a negative electrode operably coupled to the Franz cell; and
(d) an electrical current application device operably coupled to the positive and negative electrodes.

21. A method of increasing a delivery of a substance into a tissue treatment area comprising:
applying an electrical current to the tissue treatment area at a level and polarity and for a duration sufficient to enhance the transport or absorption of the substance into the tissue treatment area after the current is no longer being applied; and
placing the substance on the tissue treatment area and leaving it in contact with the treatment area after the current is no longer being applied.

22. The method according to claim 21 wherein the step of applying a current to the tissue treatment area comprises applying a current with a polarity that is substantially the same as the net charge of an active molecule of the substance.

23. The method according to claim 21 wherein the step of applying a current to the tissue treatment area comprises applying a current of at least about 2 mAmp-minutes.

24. The method according to claim 21 wherein the step of applying a current to the tissue treatment area comprises applying a current of at least about 2 mAmp-minutes in at least one cycle.

25. The method according to claim 21 wherein the step of placing the substance comprises placing a substance selected from the group consisting of a liquid, a gel, an ointment, a powder, a lotion, a foam, a solution, and a cream.

26. The method of claim 21 wherein the step of placing the substance comprises placing a substance selected from the group consisting of a body shaping gel, TruFace Line Corrector™, TruFace Priming Solution™, TruFace Revealing Gel™, Clear Action Day Treatment™, 180 Cell Renewal Fluid™, and Celltrex CoQ10 Complete™.

27. The method according to claim 21 wherein the step of applying a current to the tissue treatment area comprises applying a current to an area that has cellulite in the tissue.

28. The method according to claim 21 wherein the step of applying a current to the tissue treatment area comprises applying a current to an area that has cellulite in the tissue and the step of placing the substance comprises placing a substance that aids in cellulite removal.

29. The method according to claim 21 wherein the step of placing the substance comprises placing a substance that provides some beneficial effect to the tissue treatment area.

30. The method according to claim 29 wherein the substance treats a condition selected from the group consisting of wrinkles or lines, discoloration, redness, irritated skin, oily skin, and acne.

31. The method according to claim 29 wherein the substance aids in exfoliating skin.
